(19) United States
(12) Patent Application Publication
(10) Pub. No.: US 2014/0303135 A1
(43) Pub. Date: Oct. 9, 2014

(54) Personal Care Compositions Having Dried Zinc Pyrithione-Polymer Aggregates

(71) Applicant: THE PROCTER & GAMBLE COUNSEL, Cincinnati, OH (US)

(72) Inventors: Edward Dewey SMITH, III, Mason, OH (US); Jason Edward COOK, Anderson Township, OH (US); Mark William HAMERSKY, Indian Springs, OH (US)

(73) Assignee: The Procter & Gamble Company, Cincinnati, OH (US)

(21) Appl. No.: 13/856,457

(22) Filed: Apr. 4, 2013

(51) Int. Cl. A61K 8/02; A61K 8/58

(52) U.S. Cl. CPC A61K 8/0275; A61K 8/58
USPC 514/188

(57) ABSTRACT

Personal care composition and methods are included with respect to a dried zinc pyrithione-polymer aggregate.

### FIELD

The present disclosure generally relates to personal care compositions comprising dried zinc pyrithione-polymer aggregates and methods relating thereto.

### BACKGROUND

Human health is impacted by many microbial entities or microbials such as germs, bacteria, fungi, yeasts, molds, viruses, or the like. For example, invasion by microbial entities or microbials including various viruses and bacteria cause a wide variety of sicknesses and ailments. To reduce such an invasion, people frequently wash their skin with antimicrobial soaps. Antibacterial soaps typically include soaps in combination with, for example, antimicrobial agents. For example, one such antibacterial soap is a bar soap. When the skin is washed with an antimicrobial soap, such as a bar soap, the surfactancy of the soap typically removes most of the microbial entities or microbials on the skin, while the antimicrobial agent deposits at least in part onto the skin to provide residual protection against subsequent invasion. As such, it is desirable to improve the properties of an antimicrobial agent and/or composition to provide improved benefits.

### SUMMARY

A personal care composition comprises a dried zinc pyrithione-polymer aggregate.

A personal care composition, comprising water; from about 0.05% to about a dried zinc pyrithione-polymer aggregate having a particle size of about 1500 μm or less; and at least one of a surfactant and soap.

### DETAILED DESCRIPTION OF THE INVENTION

### I. Definitions

As used herein, the following terms shall have the meaning specified thereafter:

“Aggregate” refers to a component or composition including at least two particles joined together by a binder. For example, the joining of two particles of the same material, like zinc pyrithione.

“Anhydrous” refers to those compositions, and components thereof, which are substantially free of water.

“Bar soap” refers to solid compositions intended for topical application to a surface such as skin or hair to, for example, remove dirt, oil, and the like. The bar soaps can be rinse-off formulations. Bar soaps can be in the form of a solid (e.g., non-flowing) bar soap intended for topical application to skin. The bar soap can also be in the form of a soft solid which is conformable to the body. The bar soap additionally can be wrapped in a substrate which remains on the bar during use. Bar soap can be in any suitable form including, for example, powder, pellets, or a shaped bar (e.g. oval, circular, square, rectangular, etc.).

“Dried zinc pyrithione-polymer aggregate” refers to multiple particles of zinc pyrithione held together by a polymer, wherein the aggregate has a moisture content of about 25% or less, by weight of the aggregate.

“Personal care composition” refers to compositions intended for topical application to skin or hair. The personal care compositions can be, for example, in the form of a liquid, semi-liquid cream, lotion, gel, or solid and are intended for topical application to the skin and/or hair. Examples of personal care compositions can include but are not limited to bar soaps, shampoos, conditioning shampoos, body washes, moisturizing body washes, shower gels, skin cleansers, cleansing milks, in shower body moisturizers, pet shampoos, shaving preparations, etc.

“Polymer” refers to a macromolecule comprising repeating units. The polymer can be natural or synthetic. To exhibit polymeric properties, the polymer can have a molecular weight large enough to entangle and/or a crystallinity which allows it to entangle.

“Rinse-off” means the intended product usage includes application to skin and/or hair followed by rinsing and/or wiping the product from the skin and/or hair within a few seconds to a few minutes of the application step.

“STnS” refers to sodium trideceth(n) sulfate, wherein n can define the average number of moles of ethoxylate per molecule.

“Structured” refers to having a rheology that can confer stability on the personal care composition. A degree of structure can be determined by characteristics determined by one or more following methods: Young's Modulus Method, Yield Stress Method, or Zero Shear Viscosity Method or by a Ultracentrifugation Method, all described in U.S. Pat. No. 8,158,566, granted on Apr. 17, 2012. A cleansing phase can be considered to be structured if the cleansing phase has one or more following characteristics: (a) Zero Shear Viscosity of at least 100 Pascal-seconds (Pa-s), at least about 200 Pa-s, at least about 500 Pa-s, at least about 1,000 Pa-s, at least about 1,500 Pa-s, or at least about 2,000 Pa-s; (b) A Structured Domain Volume Ratio as measured by the Ultracentrifugation Method, of greater than about 40%, at least about 45%, at least about 50%, at least about 55%, at least about 60%, at least about 65%, at least about 70%, at least about 75%, at least about 80%, at least about 85%, or at least about 90%; or (c) A Young's Modulus of greater than about 2 Pascals (Pa), greater than about 10 Pa, greater than about 20 Pa, greater than about 30 Pa, greater than about 40 Pa, greater than about 50 Pa, greater than about 75 Pa, or greater than about 100 Pa.

“Substantially free of” refers to about 2% or less, about 1% or less, or about 0.1% or less of a stated ingredient. “Free of” refers to no detectable amount of the stated ingredient or thing.

“Water activity” refers to the relative availability of water to participate in physicochemical interactions. For example, a bar soap can exhibit a water activity of about 0.95 or less; about 0.9 or less; from about 0.4 to about 0.85; or from about 0.8 to about 0.85.

### II. Dried Zinc Pyrithione-Polymer Aggregates and Personal Care Compositions

People today tend to be more cognizant of germs and the spread of bacteria. As such, we have seen a rise in the use of antibacterial products (e.g., soap). While these products may have some antibacterial properties, people continue to be interested in products that will provide improved antimicrobial efficacy. Some properties that drive efficacy include, for example, an ability to deposit an antimicrobial agent onto the skin of a user; an amount of the antimicrobial agent deposited; and bioavailability of the antimicrobial agent.

Thus, current antibacterial cleansing products can be more effective if such products can be improved to deposit more of the antimicrobial agent (e.g., zinc pyrithione) onto the skin or if the bioavailability of what is deposited on the skin can be increased. This will result in more effective reduction of the population of microbials on the skin. Additionally, this will allow the product to effectively protect against, for example, subsequent invasion by an increasing number of microbial entities or microbials or even gram negative bacteria such as E. coli, gram positive bacteria, and the like.

One way of improving deposition of an antimicrobial agent is to form it into an antimicrobial polymer aggregate. For example, and as discovered here, zinc pyrithione can be combined with a polymer and dried to form a dried zinc pyrithione-polymer aggregate. Such dried zinc pyrithione-polymer aggregates can also be effective in increasing the antimicrobial efficacy on the surface, thereby improving protection against subsequent invasion of microbials on the surface. This allows for the use of less of the antimicrobial agent itself while at least maintaining the efficacy at the higher amount. Thus, dried zinc pyrithione-polymer aggregates can be effective in increasing the efficiency on, for example, a mass basis of the amount of zinc pyrithione deposited on the surface of the skin and/or hair of an individual. Additionally, dried zinc pyrithione-polymer aggregates can be effective at improving dust control.

Polymers have previously been introduced into personal care compositions, for example, as dissolved within a suspension. Traditionally, inclusion of polymers in personal care compositions by such a method can interfere with bioavailability of antimicrobial agents (e.g., zinc pyrithione) because they become at least partially encapsulated by the polymer. Though it is believed that increased deposition can be achieved by using polymers to form a coacervate, it is further believed that coacervates can effectively entrain the antimicrobial agent, thereby limiting an ability of the antimicrobial agent to interact with a target (e.g., fungi, bacteria, epidermis) and thus limiting its bioavailability. As formation of the coacervate can be dependent upon ionic interactions, it is believed that efficacy can be hindered as a result of the ionic interactions between the polymer and an antimicrobial agent.

Without wishing to be bound by theory, it is believed that combining and drying an antimicrobial agent (e.g., zinc pyrithione) with an added polymer (e.g., non-ionic, cationic, hydrophobic polymers), can prevent such added polymer used to form the dried polymer aggregate from interfering with the bioavailability of the antimicrobial agent (e.g., zinc pyrithione). These added polymers can also act as binders, or excipients. Generally, binders can assist in fusing primary particles into aggregate particles and affording control over properties of the aggregate particles.

Further, a dried zinc pyrithione-polymer aggregate used in personal care compositions can have a respective primary particle size, an aggregate particle size, a compressive strength, and/or a frangibility to increase efficacy, deposition, and dust control. In particular, an aggregate particle can more readily engage a surface of the skin of an individual, but as the aggregate particle breaks apart into primary particles, the primary particles can be more readily deposited on the skin, thus enhancing deposition of an antimicrobial agent (e.g., zinc pyrithione). In addition, including a polymer to form an aggregate increases the surface area, and thus can increase its respective bioavailability and its efficacy. It is believed the aggregate, for example a dried zinc pyrithione-polymer aggregate, can have an increased surface area due at least in part to its structure containing void spaces.

Compressive strength of a polymer aggregate (e.g., dried zinc pyrithione-polymer aggregate) can increase as an amount of polymer increases. See, for example, Table 7 below, which shows strength values for rehydrated dried zinc pyrithione polymer aggregate. Compressive strength is important as it is predictive of whether the polymer aggregate will hold up during processing into a final product. Aggregate particles with a polymer can possess a compressive strength such that the aggregate particles can be durable and survive processing into a personal care composition. However, the aggregate particles can also possess frangibility such that abrasive forces used during application to the skin and/or hair can release the primary particles from the aggregate particles.

TABLE 7

| | | | | Compressive strength | Compressive strength | |
|---|---|---|---|---|---|---|
| | | | ZPT:Added | (Thickness at 5 N | (Thickness at 5 N | Wet Strength |
| | | | Polymer | applied | applied | Ratio |
| | Drying Procedure | Added Polymer | Ratio | force) DRY | force) WET | (WET/DRY) |
| Inventive | Spray Dried (particles | AM:Triquat | 99:1 | 128.0 microns | 78.8 microns | 62% |
| Example 8 | recovered from cyclone) | | | | | |
| Inventive | Tray Dried | AM:Triquat | 90:10 | 465 microns | 78.4 microns | 17% |
| Example 4A | | | | | | |
| Inventive | Spray Dried (particles | AM:Triquat | 99:1 | 147 microns | 117 microns | 80% |
| Example 8 | recovered from bottom) | | | | | |
| Inventive | Spray Dried | Hydroxypropyl- | 99:1 | 244 microns | 174 microns | 71% |
| Example 10B | | cellulose (Klucel) | | | | |
| Inventive | Spray Dried | Guar hydroxypropyl- | 99:1 | 278 microns | 188 microns | 68% |
| Example 10C | | trimonium chloride | | | | |
| Comparative | Spray Dried | None | 100:0 | 193 microns | 15.5 microns | 8% |
| Example 2A | | | | | | |

The compressive strength of an aggregate (e.g., dried zinc pyrithione-polymer aggregate) can also vary based on the moisture content of the aggregate. The compressive strength can be measured as described below. Compressive strength is best measured as the aggregate exists in the final product, like a body wash. However, in some final product forms it will be difficult to measure this for the aggregate as it will be difficult to isolate the aggregate from the final form. For these forms, like bar soap, it is best to measure the compressive strength of the aggregate prior to its incorporation into the final product. Compressive strength can be expressed in terms of a thickness of a layer of aggregate particles defining a distance between an upper geometry and a glass slide of a rheometer, where a 5.0 Newton force is applied to the layer. Thus, the thickness of the layer after the application of the force as described in the compressive strength method is the compressive strength (μm). Suitable compressive strength thicknesses can go, for example, from about 50 microns to about 2,500 microns; about 60 microns to about 1,000 microns; about 70 microns to about 500 microns; about 100 microns to about 300 microns; about 125 microns to about 200; about 150 microns to about 175; or any combination thereof.

Table 9, shown below, illustrates an increase in efficacy for a dried zinc pyrithione-polymer aggregate, relative to a formulation comprising 0.5% Fine Particle Size (FPS) zinc pyrithione (Comparative Example 11). The data is from the pig skin residual efficacy test (described below) and includes log cfu reduction measurements following a five-hour incubation period. While Comparative Example 12 and Inventive Example 14 show similar results, as indicated in Table 9, Inventive Example 14 includes a polymer which can provide additional benefits, such as making the aggregate non-ionic. In particular, treatment with a bar soap comprising 0.5% of a dried zinc pyrithione-polymer aggregate, wherein the polymer is a cationic polymer, exhibited the highest efficacy. Accordingly, a method for increasing the efficacy of zinc pyrithione comprises combining zinc pyrithione with a polymer and drying the zinc pyrithione polymer combination to form an aggregate.

TABLE 9

| | Log cfu Reduction vs. Placebo |
|---|---|
| Comparative Example 11 | 1.47 |
| Inventive Example 14 | 1.69 |
| Comparative Example 12 | 1.70 |
| Inventive Example 15 | 1.83 |

Table 10, also shown below, shows the results for a cup scrub test measuring the deposition of zinc pyrithione on a pig skin following treatment of those skins with a bar soap of Comparative Examples 11 and 12 and Inventive Example 14. As illustrated, the formulation comprised of a spray-dried zinc pyrithione-polymer aggregate with a non-ionic polymer (Inventive Example 14) provided enhanced deposition of the antibacterial ingredient to the skin relative to colloidal zinc pyrithione and a formulation comprising spray-dried zinc pyrithione. Accordingly, a method for enhancing the deposition of zinc pyrithione comprises combining zinc pyrithione with a polymer and drying the zinc pyrithione polymer combination to form an aggregate. The zinc pyrithione composition may be in the form of a suspension prior to combination with the polymer. The polymer may be in the form of a solution prior to combination with the zinc pyrithione. The zinc pyrithione-polymer aggregate may have a moisture content of about 25% or less, by weight of the aggregate. The zinc pyrithione-polymer aggregate may comprise from about 0.1% to about 20% of polymer, by weight of the aggregate, of the polymer, and from about 80% to about 99.9%, by weight of the aggregate, of zinc pyrithione. The zinc pyrithione polymer combination may be spray dried to form the aggregate.

Another method for enhancing deposition of zinc pyrithione comprises applying a personal care composition including a dried zinc pyrithione-polymer aggregate which can provide a deposition from about 0.01 μg/cm² to about 5 μg/cm² of zinc pyrithione to the skin. The personal care composition may further provide deposition of from about 0.10 μg/cm² to about 1 μg/cm² of zinc pyrithione to the skin; from about 0.35 μg/cm² to about 0.5 μg/cm² of zinc pyrithione to the skin; or from about 0.4 μg/cm² to about 0.45 μg/cm² of zinc pyrithione to the skin.

TABLE 10

Zinc Pyrithione
Deposition (μg/cm²)

| | Mean | SE |
|---|---|---|
| Comparative Example 11 | 0.160 | 0.032 |
| Comparative Example 12 | 0.335 | 0.066 |
| Inventive Example 14 | 0.438 | 0.080 |

As noted herein, the amount and type of polymer used as a binder with the dried zinc pyrithione-polymer aggregate do not adversely affect antimicrobial efficacy. This surprising result of having a polymer added to zinc pyrithione to form an aggregate, for instance, and still providing the antimicrobial efficacy as illustrated herein provides an unexpected alternative in forming and using antimicrobial agents and actives. Further, varying the method of drying, an aggregate particle size, and/or shape also do not appear to adversely affect antimicrobial efficacy. For example, Table 8 illustrates minimum concentrations of an active ingredient required to inhibit microbial growth in a solution-based efficacy test. And as shown, the amount of active ingredient required to inhibit microbial growth remains relatively constant even as the amount of polymer, the type of polymer, and the drying method are varied. Further, increasing levels of a cationic polymer can be used in combination with an active without experiencing a decrease in antimicrobial efficacy, where such active can exhibit a surface charge. Thus, a polymer can be used as a binder with zinc pyrithione to provide added benefits without experiencing a decrease in antimicrobial efficacy or bioavailability. Such results provided added benefits when selecting other ingredients to be included with a dried zinc pyrithione-polymer aggregate to form a personal care composition.

TABLE 8

| Active Ingredient Form | E. Coli MIC (ppm) | S. aureus MIC (ppm) |
|---|---|---|
| Comparative Example 1 | 3.125-12.5 | 6.25-12.5 |
| Inventive Example 4A | 6.25-12.5 | 6.25-12.5 |
| Inventive Example 5A | 12.5 | 12.5 |
| Inventive Example 6A | 12.5 | 12.5 |
| Inventive Example 7 | 6.25 | 6.25 |
| Inventive Example 8 | 6.25 | 6.25 |
| Inventive Example 10A | 6.25 | 6.25 |

As mentioned above, a method of forming a dried polymer aggregate to improve dust control comprises precipitating an antimicrobial agent with a polymer and drying the precipitated polymer aggregate. Dust control can be effected by aggregating fine particles which normally generate dust during manufacturing (e.g., by conveying, mixing, weighing). Further, durable aggregate particles can prevent dust creation that can be caused by agitation, for example, during shipping. The dried zinc pyrithione-polymer aggregates can be too heavy to become entrained in air, which can be problematic, for safety reasons, with actives and antimicrobial agents. Thus, the fine particles do not tend to generate dust when formed as part of the aggregate.

Generally, dust control can be achieved via increased aggregate particle size. It is believed that dust control can be effected by virtue of the aggregate particles have a greater mass on a per-particle basis, thereby requiring a greater mechanical input to impart an inertia sufficient to send the aggregate particles to a headspace. Further, the aggregate particles can also be less affected by convective currents in a vicinity relative to powders having less mass. It is also believed that a risk of inhalation can be reduced as the aggregate particle size increases. Though an ideal particle size for achieving maximum efficacy can resemble that of dust, by using the dried zinc pyrithione-polymer aggregate in the personal care composition, an aggregate particle size can be increased above a range associated with the risk of inhalation, and the primary particles, which can have a primary particle size conducive for maximum efficacy, can be released from the aggregate particles by shear force during application of the personal care composition.

Conventional zinc pyrithione can be made, for example, by reacting 1-hydroxy-2-pyridinethione (i.e., pyrithione acid) or a soluble salt thereof with a zinc salt (e.g. zinc sulfate) to form a zinc pyrithione precipitate as illustrated in U.S. Pat. No. 2,809,971, and the zinc pyrithione can be formed or processed into platelets using, for example, sonic energy as illustrated in U.S. Pat. No. 6,682,724. These processes, however, do not include drying. Conventional zinc pyrithione is an aqueous suspension and one example of a conventional ZPT, FPS, has a moisture content of about 52%.

Conventional zinc pyrithione can be combined with a polymer in solution and dried to form a dried zinc pyrithione-polymer aggregate. The dried zinc pyrithione-polymer aggregate can be formed from one or more of a variety of drying processes. Examples of such drying processes can include, but are not limited to spray drying, tray drying, tunnel drying, roller drying, fluidized bed drying, pneumatic drying, rotary drying, trough drying, bin drying, belt drying, vacuum drying, drum drying, infrared drying, microwave drying, and radiofrequency drying.

Polymers suitable for use herein as part of an aggregate can include, for example, non-ionic, cationic, and anionic polymers. Suitable polymers can also include oligomers, copolymers, including random copolymers and organized copolymers such as block polymers, hydrophobic polymers, hydrophobically modified polymers, water soluble polymers, water insoluble polymers, natural polymers, synthetic polymers, and emulsion polymers such as latex polymers (e.g. ACUSOL™ OP301), hot melt polymers, and adhesive polymers.

Examples of such non-ionic polymers can include cellulosic-based polymers such as cellulose, microfibrous cellulose, hydroxypropyl methylcellulose (HPMC), hydroxypropyl cellulose, hydroxyethyl cellulose, and mixtures thereof. Other such suitable non-ionic polymers can include natural hydrocolloids such as xanthan gum, polyvinyl alcohol, thermally-activated polymers such as ethylenevinylacetate copolymers, silicone polymers and adhesives, polyvinyl alcohols, and polymers having the following formula:

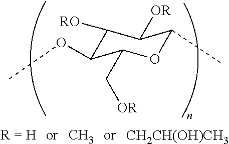

Suitable cationic polymers that can be used in combination with zinc pyrithione to form polymer aggregates can include, for example, acrylamide-based polymers such as an acrylamide triquat copolymers (“AM:Triquat” or Polyquaternium 76). Acrylamide copolymers can be synthesized using monomers that contain three quaternary nitrogen groups. Suitable cationic polymers can also include cationic guars such as guar hydroxypropyltrimonium chloride, cationic celluloses such as polyquaternium 10 and polyquaternium 67, polydiallyldimethylammonium chloride, and cationic starches.

Suitable hydrophobic polymers for use herein can include, for example, polyisobutylene, petrolatum, microcrystalline wax, a material selected from the group consisting of poly(isobutylene), a copolymer comprising poly(isobutylene), poly(ethylene), a copolymer comprising poly(ethylene), poly(propylene), a copolymer comprising poly(propylene), poly(butadiene), a copolymer comprising poly(butadiene), poly(isoprene), a copolymer comprising poly(isoprene), hydrogenated poly(butadiene), a copolymer comprising hydrogenated poly(butadiene), hydrogenated poly(isoprene), a copolymer comprising hydrogenated poly(isoprene), poly(alpha-olefin), a copolymer comprising poly(alpha-olefin), or combinations thereof.

A dried zinc pyrithione-polymer aggregate can include about 0.1% or more of a polymer; about 1% or more of a polymer; about 5% or more of a polymer; about 10% or more of a polymer; or about 20% or more of a polymer, by weight of the dried zinc pyrithione-polymer aggregate, assuming the utilized amount does not encapsulate the zinc pyrithione and thus reduce its efficacy. Further, an amount of polymer included within the dried zinc pyrithione-polymer aggregate can be determined with respect to virgin raw material zinc pyrithione Zinc pyrithione can include trace components, and the amount of polymer included within dried zinc pyrithione-polymer aggregate can refer to an amount of polymer added to the zinc pyrithione to form the aggregate, and is not intended to include such trace components.

Without wishing to be bound by theory, if a percentage of polymer in an aggregate is too high, the dried zinc pyrithione-polymer aggregate can encapsulate the zinc pyrithione particles, hardening the aggregate and reducing efficacy. However, if there is no polymer, the aggregate can easily disperse in water such that there is no aggregate. Thus, it may be determined that a certain amount of polymer performs better in the formation of an aggregate. For example a zinc pyrithione-polymer aggregate may comprise from about 0.5% to about 10% of a polymer. Accordingly, the corresponding dried zinc pyrithione-polymer aggregate could include from about 90% to about 99.5% of zinc pyrithione, by weight of the dried zinc pyrithione-polymer aggregate. Other exemplary amounts of zinc pyrithione in an aggregate include, for example, from about 92% to about 99% of zinc pyrithione; from about 95% to about 99% of zinc pyrithione; or from about 98% to about 99% of zinc pyrithione, by weight of the dried zinc pyrithione-polymer aggregate.

Optional secondary actives, excipients, stabilizing agents, e.g., water-insoluble particles or domains, can be combined with zinc pyrithione and a polymer and dried to form a dried polymer aggregate. For example, such optional actives can include metal carbonates (e.g., potassium carbonate), zinc oxide, trichlorocarbanilide, selenium disulfide, octopirox (MEA salt of 1-hydroxy-4-methyl-6-(2,4,4-trimethylpentyl)-2(1H)-pyridone. Additionally, the optional secondary ingredient could comprise a surfactant. Suitable surfactants could include those listed below. Without being limited by theory, it is believed the use of a surfactant with a charge opposite to that of the particle will help control the solubility of the finished aggregate and give better results due to the decrease of the solubility of the particle when an oppositely charged surfactant is bound thereto. Optional secondary actives could be included, for example, in an amount of 95% or less, by weight of the aggregate; from about 5% to about 90%; from about 10% to about 70%, or from about 20% to about 60%, by weight of the dried polymer aggregate.

Primary particles of the dried zinc pyrithione-polymer aggregate can be in the form of particulates, platelets, or a combination thereof, for example. Primary particles can comprise an average primary particle size from about 0.02 micron to about 25 microns; from about 0.5 micron to about 1 mm; or from about 0.7 micron to about 4 microns. Aggregate particulates can comprise an aggregate mean particle size from about 0.1 micron to about 5 mm; from about 0.5 micron to about 10 microns; from about 0.75 micron to about 5 microns; or any combination thereof. Aggregate particle size can be determined by conventional light scattering techniques for powders, for example, using a Malvern Mastersizer, or by microscopy. The aggregate particles can have various shapes, including, for example, spheres, rods, discs, cubes, or a combination thereof. Spherical aggregate particles may provide added benefits.

Dried zinc pyrithione-polymer aggregate may be dried with excipients such as metallic carbonates, auxiliary active such as selenium compounds, organic actives such as triclosan or trichlorocarbanilide, acidic or basic actives, and the like, wherein such excipients can enhance bioactivity. Aggregate particles may be formed so as to contain no internal porosity or with void spaces to have a high internal porosity such that the aggregate particles can maintain properties relating to surface area.

The moisture content of a dried zinc pyrithione-polymer aggregate can be controlled. A suitable moisture level may include about 25% or less, by weight of the dried zinc pyrithione-polymer aggregate. The dried zinc pyrithione-polymer aggregate may have an even lower moisture content, for example by being dried further, and that moisture content could be 22%, 20, 18, 15, 12, 10, 8, 6, 5, 3, or 1%, or less, by weight of the dried zinc pyrithione-polymer aggregate. While some types of drying are exemplified herein, any appropriate method to reduce moisture level can be used. In soap compositions, for example, the aggregate particles have to be durable to survive milling. If the moisture content is too high, the aggregate particles won't be sufficiently durable to survive such processing.

### Personal Care Composition

A personal care composition can include a dried zinc pyrithione-polymer aggregate. The zinc pyrithione-polymer aggregate may be present from about 0.01% to about 5%, by weight of the personal care composition. It may be present at even smaller amounts like from about 0.05% to about 2%, from about 0.1% to about 2%, about 0.5%, about 1.0% or more, or any combination thereof, by weight of the personal care composition, for example.

Many personal care compositions can be water-based. As such, a personal care composition can include from about 0.1% to about 35%, from about 0.3% to about 20%, or about 10%, by weight of the personal care composition, of water. It should be understood that an amount of water can be lost, i.e. evaporated, during a process of making a personal care composition, or subsequently, with water being absorbed by surrounding packaging (e.g. a cardboard carton), and the like. Thus, a personal care composition can also include materials that tend to bind the water such that the water can be maintained in the personal care composition at the desired levels. Examples of such materials can include carbohydrate structurants and humectants such as glycerin. However, it will be appreciated that a personal care composition can be anhydrous.

A variety of optional ingredients can also be added to a personal care composition. Such suitable ingredients can include, but are not limited to, structurants, humectants, fatty acids, inorganic salts, and other antimicrobial agents or actives.

A personal care composition can also optionally include hydrophilic structurants such as carbohydrate structurants and gums. Some suitable carbohydrate structurants include raw starch (corn, rice, potato, wheat, and the like) and pregelatinized starch. Some suitable gums include carrageenan and xanthan gum. A personal care composition may include from about 0.1% to about 30%, from about 2% to about 25%, or from about 4% to about 20%, by weight of the personal care composition, of a carbohydrate structurant. A personal care composition can also optionally include one or more humectants.

Examples of such humectants can include polyhydric alcohols. Further, humectants such as glycerin can be included the personal care composition as a result of production or as an additional ingredient. For example, glycerin can be a by-product after saponification of the personal care composition. Including additional humectant can result in a number of benefits such as improvement in hardness of the personal care composition, decreased water activity of the personal care composition, and reduction of a weight loss rate of the personal care composition over time due to water evaporation.

A personal care composition can optionally include inorganic salts. Inorganic salts can help to maintain a particular water content or level of the personal care composition and improve hardness of the personal care composition. The inorganic salts can also help to bind the water in the personal care composition to prevent water loss by evaporation or other means. A personal care composition can optionally include from about 0.01% to about 15%, from about 1% to about 12%, or from about 2.5% to about 10.5%, by weight of the personal care composition, of inorganic salt. Examples of suitable inorganic salts can include magnesium nitrate, trimagnesium phosphate, calcium chloride, sodium carbonate, sodium aluminum sulfate, disodium phosphate, sodium polymetaphosphate, sodium magnesium succinate, sodium tripolyphosphate, aluminum sulfate, aluminum chloride, aluminum chlorohydrate, aluminum-zirconium trichlorohydrate, aluminum-zirconium trichlorohydrate glycine complex, zinc sulfate, ammonium chloride, ammonium phosphate, calcium acetate, calcium nitrate, calcium phosphate, calcium sulfate, ferric sulfate, magnesium chloride, magnesium sulfate, and tetrasodium pyrophosphate.

A personal care composition can optionally further include one or more additional antibacterial agents that can serve to further enhance antimicrobial effectiveness of the personal care composition. A personal care composition can include, for example, from about 0.001% to about 2%, from about 0.01% to about 1.5%, or from about 0.1% to about 1%, by weight of the personal care composition, of additional antibacterial agent(s). Examples of suitable antibacterial agents can include carbanilides, triclocarban (also known as trichlorocarbanilide), triclosan, a halogenated diphenylether available as DP-300 from Ciba-Geigy, hexachlorophene, 3,4,5-tribromosalicylanilide, and salts of 2-pyridinethiol-1-oxide, salicylic acid, and other organic acids. Other suitable antibacterial agents are described in U.S. Pat. No. 6,488,943.

Solid Cleansing Composition

As noted herein, personal care compositions can take on numerous forms. One suitable form is that of a solid personal care composition. Solid compositions can take on many forms like powder pellets, bars, etc. These forms will generally be described as bar soap, but it should be understood the solid composition could be in another form or shape. One example of a bar soap personal care composition can include from about 0.1% to about 35%, by weight of the personal care composition, of water, from about 45% to about 99%, by weight of the personal care composition, of soap, and from about 0.01% to about 5%, by weight of the personal care composition, of dried zinc pyrithione-polymer aggregate. Another suitable antimicrobial bar soap can include, for example, from about 0.1% to about 30%, by weight of the personal care composition, of water, from about 40% to about 99%, by weight of the personal care composition, of soap, and from about 0.01% to about 1%, by weight of the personal care composition, of dried zinc pyrithione-polymer aggregate.

A dried zinc pyrithione-polymer aggregate can be treated before being used in an antimicrobial bar soap. For example, a dried zinc pyrithione-polymer aggregate can be stabilized, for example, against flocculation. A dried zinc pyrithione-polymer aggregate (e.g., particulate and/or platelet form) used in an antimicrobial bar soap can have a surface modification thereon to prevent the particulates and/or platelets from attaching to each other. Further, the surface modification can aid in formation the dried zinc pyrithione-polymer aggregate by charge association (e.g., cationic polymer association with anionic particle surface). The surface modification can include, for example, polynaphthalene sulfonate or any other suitable sulfate, sulfonate, carboxylate, or other compound that provides stability, for example, by charge or steric barrier on a surface. The treatment can be applied to the aggregate surface or to the surface of the zinc pyrithione particle prior to forming the aggregate.

Bar soap compositions can be referred to as conventional solid (i.e. non-flowing) bar soap compositions. Some bar soap composition comprise convention soap, while others contain synthetic surfactants, and still others contain a mix of soap and synthetic surfactant. Bar compositions may include, for example, from about 0% to about 45% of a synthetic anionic surfactant. An example of a suitable conventional soap can include milled toilet bars that are unbuilt (i.e. include about 5% or less of a water-soluble surfactancy builder).

A personal care bar composition can include, for example from about 45% to about 99% or from about 50% to about 75%, by weight of the personal care composition, of soap. Such soaps can include a typical soap, i.e., an alkali metal or alkanol ammonium salt of an alkane- or alkene monocarboxylic acid. Sodium, magnesium, potassium, calcium, mono-, di- and tri-ethanol ammonium cations, or combinations thereof, can be suitable for a personal care composition. The soap included in a personal care composition can include sodium soaps or a combination of sodium soaps with from about 1% to about 25% ammonium, potassium, magnesium, calcium, or a mixture of these soaps. Additionally, the soap can be well-known alkali metal salts of alkanoic or alkenoic acids having from about 12 to about 22 carbon atoms or from about 12 to about 18 carbon atoms. Another suitable soap can be alkali metal carboxylates of alkyl or alkene hydrocarbons having from about 12 to about 22 carbon atoms. Additional suitable soap compositions are described in U.S. patent application Ser. No. 13/036,889.

A personal care composition can also include soaps having a fatty acid. For example, one bar soap composition could use from about 40% to about 95% of soluble alkali metal soap of C₈-C₂₄ or C₁₀-C₂₀ fatty acids. The fatty acid may, for example, have a distribution of coconut oil that can provide a lower end of a broad molecular weight range or a fatty acid distribution of peanut or rapeseed oil, or their hydrogenated derivatives, which can provide an upper end of the broad molecular weight range. Other such compositions can include a fatty acid distribution of tallow and/or vegetable oil. The tallow can include fatty acid mixtures that can typically have an approximate carbon chain length distribution of 2.5% C₁₄, 29% C₁₆, 23% C₁₈, 2% palmitoleic, 41.5% oleic, and 3% linoleic. The tallow can also include other mixtures with a similar distribution, such as fatty acids derived from various animal tallows and/or lard. In one example, the tallow can also be hardened (i.e., hydrogenated) such that some or all unsaturated fatty acid moieties can be converted to saturated fatty acid moieties.

Suitable examples of vegetable oil include palm oil, coconut oil, palm kernel oil, palm oil stearine, soybean oil, and hydrogenated rice bran oil, or mixtures thereof, since such oils can be among more readily available fats. One example of a suitable coconut oil can include a proportion of fatty acids having at least 12 carbon atoms of about 85%. Such a proportion can be greater when mixtures of coconut oil and fats such as tallow, palm oil, or non-tropical nut oils or fats can be used where principle chain lengths can be C₁₆ and higher. The soap included in a personal care composition can be, for example, a sodium soap having a mixture of about 67-68% tallow, about 16-17% coconut oil, about 2% glycerin, and about 14% water.

Soap included in a personal care composition can also be unsaturated in accordance with commercially acceptable standards. For example, a soap included in a personal care composition could include unsaturation in a range of from about 37% to about 45% of saponified material.

Soaps included in a personal care composition can be made, for example, by a classic kettle boiling process or modern continuous soap manufacturing processes wherein natural fats and oils such as tallow or coconut oil or their equivalents can be saponified with an alkali metal hydroxide using procedures well known to those skilled in the art. Soap can also be made by neutralizing fatty acids such as lauric (C₁₂), myristic (C₁₄), palmitic (C₁₆), or stearic (C₁₈) acids, with an alkali metal hydroxide or carbonate.

Soap included in a personal care composition could also be made by a continuous soap manufacturing process. The soap could be processed into soap noodles via a vacuum flash drying process. One example of a suitable soap noodle comprises about 67.2% tallow soap, about 16.8% coconut soap, about 2% glycerin, and about 14% water, by weight of the soap noodle. The soap noodles can then be utilized in a milling process to finalize a personal care composition.

A personal care composition can also optionally include one or more free fatty acids at an amount of from about 0.01% to about 10%, from about 0.5% to about 2%, or from about 0.75% to about 1.5%, by weight of the personal care composition. Free fatty acids can be included in the personal care composition to provide enhanced skin feel benefits such as softer and smoother feeling skin. Suitable free fatty acids can include tallow, coconut, palm, and palm kernel fatty acids.

Liquid Personal Care Compositions

Personal care compositions can take on many forms and one of those suitable forms can be a liquid form. Examples of personal care compositions in liquid form can include shampoo, hand soap, body wash, hand sanitizers, etc. Such liquid-based personal care compositions can include a cleansing phase and/or a benefit phase (i.e., a single- or multi-phase composition). Each of a cleansing phase or a benefit phase can include various components. The liquid composition can have multiple phases in varying combinations. For example, a personal care composition can include two cleansing phase, a cleansing phase and a benefit phase, two benefit phases, or any acceptable combination of phases. Additionally, the phases in a multi-phase composition can be blended, separate, or a combination thereof. The phases may also form a pattern (e.g. striped). A personal care composition may be micellar, lamellar, or a combination thereof. A personal care composition could comprise at least a 70% lamellar structure. A dried ZPT may be placed in a cleansing phase.

A cleansing phase may be aqueous or anhydrous. A cleansing phase may also, for example, include alcohol. A cleansing phase may comprise a surfactant. A cleansing phase may comprise from about 0.1% to about 20%, by weight of the phase, from about 3% to about 12%, from about 10% to about 20%, or any combination thereof, of a surfactant.

Surfactants suitable for use herein include anionic, zwitterionic, amphoteric, and combinations thereof. One example of a suitable surfactant comprises sodium laureth-1 sulfate, such that the dried zinc pyrithione can be used in a micellar body wash, which is described in greater detail below. A cleansing phase can also comprise at least one of an amphoteric surfactant and a zwitterionic surfactant. Suitable amphoteric or zwitterionic surfactants can include, for example, those described in U.S. Pat. No. 5,104,646 and U.S. Pat. No. 5,106,609, and those described below as cosurfactants.

A cleansing phase may include an aqueous structured surfactant phase, such that from about 5% to about 20%, by weight of the personal care composition, is a structured surfactant phase. Such a structured surfactant phase can include, for example, sodium trideceth(n) sulfate, hereinafter STnS, wherein n can define average moles of ethoxylation. n can range from about 0 to about 3, from about 0.5 to about 2.7, from about 1.1 to about 2.5, from about 1.8 to about 2.2, or n can be about 2. When n can be less than 3, STnS can provide improved stability, improved compatibility of benefit agents within personal care compositions, and increased mildness of the personal care compositions, such described benefits of STnS are disclosed in U.S. patent application Ser. No. 13/157,665.

A cleansing phase can also comprise a structuring system. One example of a structuring system includes a non-ionic emulsifier, an associative polymer, an electrolyte, or a combination thereof.

A personal care composition can be optionally free of sodium lauryl sulfate, hereinafter SLS. However, when SLS is present, suitable examples of SLS are described in U.S. patent application Ser. No. 12/817,786.

A personal care composition can include from about 0.1% to 20%, by weight of the personal care composition, of a cosurfactant. Cosurfactants can comprise amphoteric surfactants, zwitterionic surfactants, or mixtures thereof. Examples of suitable amphoteric or zwitterionic surfactants can include those described in U.S. Pat. No. 5,104,646 and U.S. Pat. No. 5,106,609.

Amphoteric surfactants can include those that can be broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378. Other examples of amphoteric surfactants can include sodium lauroamphoacetate, sodium cocoamphoactetate, disodium lauroamphoacetate disodium cocodiamphoacetate, and mixtures thereof. Amphoacetates and diamphoacetates can also be used.

Zwitterionic surfactants suitable for use can include those that are broadly described as derivatives of aliphatic quaternary ammonium, phosphonium, and sulfonium compounds, in which aliphatic radicals can be straight or branched chains, and wherein an aliphatic substituent can contain from about 8 to about 18 carbon atoms such that one carbon atom can contain an anionic group, e.g., carboxy, sulfonate, sulfate, phosphate, or phosphonate. Other zwitterionic surfactants can include betaines, including cocoamidopropyl betaine.

Other optional additives can be included in the cleansing phase, including for example emulsifiers (e.g., non-ionic emulsifier) and electrolyes. Suitable emulsifiers and electrolytes are described in U.S. patent application Ser. No. 13/157,665.

Personal care compositions can also include a benefit phase. The benefit phase can be hydrophobic and/or anhydrous. The benefit phase can also be substantially free of or free of surfactant. A benefit phase can also include a benefit agent. In particular, a benefit phase can comprise from about 0.1% to about 50%, by weight of the personal care composition, of a benefit agent or from about 0.5% to about 20%, by weight of the personal care composition, of a benefit agent. Examples of the benefit agent can include petrolatum, glyceryl monooleate, mineral oil, triglycerides, soybean oil, castor oil, soy oligomers, and mixtures thereof. Additional examples of benefit agents can include water insoluble or hydrophobic benefit agents. Other suitable benefit agents are described in U.S. patent application Ser. No. 13/157,665. The benefit phase may also comprise a dried zinc pyrithione.

Non-limiting examples of glycerides suitable for use as hydrophobic skin benefit agents herein can include castor oil, safflower oil, corn oil, walnut oil, peanut oil, olive oil, cod liver oil, almond oil, avocado oil, palm oil, sesame oil, vegetable oils, sunflower seed oil, soybean oil, vegetable oil derivatives, coconut oil and derivatized coconut oil, cottonseed oil and derivatized cottonseed oil, jojoba oil, cocoa butter, and combinations thereof.

Non-limiting examples of alkyl esters suitable for use as hydrophobic skin benefit agents herein can include isopropyl esters of fatty acids and long chain esters of long chain (i.e. C10-C24) fatty acids, e.g., cetyl ricinoleate, non-limiting examples of which can include isopropyl palmitate, isopropyl myristate, cetyl riconoleate, and stearyl riconoleate. Other examples can include hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as hydrophobic skin benefit agents herein can include oleyl myristate, oleyl stearate, oleyl oleate, and combinations thereof.

Non-limiting examples of polyglycerin fatty acid esters suitable for use as hydrophobic skin benefit agents herein can include decaglyceryl distearate, decaglyceryl diisostearate, decaglyceryl monomyriate, decaglyceryl monolaurate, hexaglyceryl monooleate, and combinations thereof.

Non-limiting examples of lanolin and lanolin derivatives suitable for use as hydrophobic skin benefit agents herein can include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Non-limiting examples of silicone oils suitable for use as hydrophobic skin benefit agents herein can include dimethicone copolyol, dimethylpolysiloxane, diethylpolysiloxane, mixed C1-C30 alkyl polysiloxanes, phenyl dimethicone, dimethiconol, and combinations thereof. Nonlimiting examples of silicone oils useful herein are described in U.S. Pat. No. 5,011,681. Still other suitable hydrophobic skin benefit agents can include milk triglycerides (e.g., hydroxylated milk glyceride) and polyol fatty acid polyesters.

### III. Procedures

A. Drying Techniques Used for Preparing Dried Zinc Pyrithione-Polymer Aggregate

### Spray Drying

Zinc pyrithione is obtained as a 49% active suspension of FPS Zinc OMADINE®. Binders, such as polymers, are obtained directly from their respective suppliers. Aqueous solutions of polymers are prepared and subsequently mixed with a zinc pyrithione aqueous suspension in relative proportions to deliver desired dry-weight based ratios of zinc pyrithione to a polymer such as hydroxypropyl methylcellulose (HPMC). The zinc pyrithione-polymer mixture is then spray dried using a Büchi Mini Spray Dryer B290 with an inlet temperature of 200° C. and an outlet temperature of 100° C. The slurry flow rate is controlled by adjusting the peristaltic pump control to 35% of a maximum pump speed. The compressed air flow rate for a feed dispersion is set to approximately 600 L/hr. The spray-dried zinc pyrithione aggregates are observed to have particle size of about 10 microns to about 100 microns by light microscopy with an average diameter of about 60 microns, while being comprised of distinct primary particle subunits, which are the original FPS particles. The aggregates are spherical.

### Tray Drying

Zinc pyrithione is obtained as a 49% active suspension of FPS Zinc OMADINE®. Binders, such as polymers, are obtained directly from their respective suppliers. Aqueous solutions of polymers are prepared and subsequently mixed with a zinc pyrithione aqueous suspension in relative proportions to deliver desired dry-weight based ratios of zinc pyrithione to polymer. The mixtures are then transferred to trays and dried in an oven, at a temperature of about 45° C., until solid and thoroughly dry as indicated by no further loss in weight of the tray. Aggregate material is then removed from the tray and mechanically broken into small particles using, for example, a blender. Resulting fractions are then obtained using U.S. Standard Sieves to yield aggregate particle size fractions. For example, aggregate particles having an average particle size from about 1 micron to about 20 microns are conducive for distributing an antimicrobial agent on the skin; aggregate particles having an average particle size from about 20 microns to about 150 microns can be used to apply an antimicrobial agent to the skin and not be visible in bar soap; and aggregate particles having an average particle size from about 150 microns to about 10 mm can be used to communicate a benefit to a consumer and be visible in bar soap. The particles are irregular in shape.

B. Pig Skin Residual Efficacy Test

To prepare a placebo, perform a one wash/rinse performance protocol. In particular, generate an overnight bacterial culture of E. coli (strain 10536, 8879, or 11259) by inoculating 50 mL of Tryptic Soy Broth (TSB) with one colony obtained from a Tryptic Soy Agar (TSA) streak plate. Grow the culture for 17-18 hr, 37° C., 200 rpm in a dry shaker.

To determine efficacy of a bar soap, perform bar soap ex vivo performance tests on pigskins. First, obtain, clean, refrigerate, and irradiate (25-40 kGy) the pigskins. Store the irradiated pigskins at −20° C. until testing. To test bar soap compositions, thaw 10×10 cm pigskins to room temperature for 1 hour, and cut the pigskins into 5×10 cm sections using a sterile scalpel.

Using a gloved hand, wash the pigskins as follows: Rinse a 5×10 cm pigskin for 15 seconds, with tap water at 33-36° C. with a flow rate of 4-4.2 L/min Wet the bar soap composition in the running water for 5 seconds, lay the bar composition flat on the pigskin surface, then immediately rub the bar soap composition gently across the entire pigskin surface for 15 seconds using back and forth motions and light hand pressure similar to that during conventional hand washing. Then, generate lather by continuously rubbing the pigskin for 45 seconds with the hand (e.g. absent the bar soap composition). Rinse the pigskin with tap water for 15 seconds by holding the tissue at a 45 degree angle and allowing the water to impinge on the top surface and cascade downwards across the entire surface. Lightly pat the pigskin dry with a sterile tissue, and allow the pigskin to dry for 5-10 minutes in still room air under low light conditions.

Cut the pigskin into 2×2.5 cm slices and inoculate each slice with 10⁶-10⁷ cfus by using 10 μL of a 1:20 dilution of TSB obtained from an overnight culture as described above. Allow the bacteria to dry on the slice of the pigskin surface for 20 minutes, then place the slice of the pigskin into a humidified chamber (60% RH, 33° C.), and incubate the slices for 0 hours, 2 hours, or 5 hours. After incubation, place the slice into a jar containing 50 mL of ice cold neutralization buffer of Modified Leethen Broth with 1.5% Tween-80 and 1% Lecithin (MBL-T), and vigorously shake the buffer with the slice therein for 1 minute to elute bacteria. As necessary, dilute the suspension in MBL-T and place the suspension onto TSA plates to obtain cell counts. Incubate the plates for 24 hours, at 33° C., and 60% Relative Humidity. Then, count the TSA plates (e.g. the cfus thereon) to calculate the cfu/mL and generate a growth curve using GraphPad Prism v4.1. Perform the test described above once to calculate the cfu/mL and to generate the growth curve. (Note: The test described above can also be performed multiple times and the data for each repetition can be averaged).

C. Cup Scrub Procedure for Measuring Deposition

As noted herein, the Cup Scrub Procedure is used to determine how much zinc-containing and/or pyrithione material is deposited onto a pig skin. First, wet a pig skin surface under running water (flow=4.5 L/min, temp=35-38° C.) for 15 seconds. If body wash is used, apply a dose of 1 mL (via disposable syringe) to the pig skin surface. Proceed to generate lather on the target substrate by rubbing the applied body wash by hand for 15 seconds. Wait 15 seconds. Following the 15-second lathering process, the lather is allowed to sit undisturbed on the pig skin for an additional 15 seconds. At the end of the 15-second wait (30 seconds after the start of the lathering process), rinse the pig skin for 10 seconds, allowing the running water to contact the target substrate surface and cascade down (toward the distal surface). Following the rinse, use a paper towel to gently pat the surface dry.

If bar soap is used, wet a pig skin surface under running water (flow=4.5 L/min, temp=35-38° C.) for 15 seconds. Next, wet the bar soap by rolling the bar soap in one hand under running water for 5 seconds. Rub the bar soap on the pig skin surface for 15 seconds, maintaining direct contact for the entire time. Place the bar soap to the side and proceed to generate lather on the pig skin surface by hand massaging the pig skin surface for 30 seconds. Rinse the pig skin for 10 seconds, allowing the running water to contact the target substrate surface and cascade down (toward the distal surface). Following the rinse, use a paper towel to gently pat the surface dry.

Place a 2-cm diameter glass cylinder containing a bead of silicone caulking on a skin contact edge firmly against a pig skin surface to prevent leakage of an extraction fluid. One mL of the extraction solvent is pipetted into the glass cylinder. To determine how much zinc pyrithione is deposited, for example, the extraction solvent can be 80:20 0.05 M EDTA:EtOH. While using a transfer pipette or glass rod, an entire area within the glass cylinder is scrubbed for about 30 seconds using moderate pressure. The solution is removed and pipetted into a labeled glass sample vial. The Cup Scrub Procedure is repeated using fresh extraction solution, which is pooled with the initial extraction in the labeled vial.

Samples are stored at 4° C. (±3° C.) until the samples are submitted for HPLC analysis. HPLC analysis is used to determine the amount of deposition. The free pyrithione in solution is then derivatized with 2-2′-Dithiopyridine, and subsequently analyzed via HPLC utilizing UV detection. The results are reported as μg of zinc pyrithione per mL of solution.

D. Compressive Strength Test

A normal force sensing rheometer with an 8 mm diameter flat steel plate geometry, e.g., TA Instruments (Delaware, USA) AR2000, is used to measure an aggregate at 25° C. A flat glass microscope slide is placed on the rheometer baseplate and an upper geometry is zeroed using a 1 Newton force criterion. The location of the slide on the baseplate and the geometry against the slide is marked before raising the upper geometry using a permanent marker. The slide is removed to a balance, and 1.0 mg of aggregates is carefully added to the area of the slide where the baseplate contacted. The slide is returned to the baseplate in its prior location. The aggregates are carefully spread to a relatively even layer using the edge of a small spatula. The upper geometry is moved to a position above the aggregates and programmed to descend at a rate of 10 microns per second to a final position 10 microns above the glass slide, collecting one datum per second. A termination criterion of 35 Newton force is programmed. The normal force is tared to zero and the rheometer is instructed to proceed with the measurement. Results are interpolated to obtain the gap between the upper geometry and glass slide in microns when a 5.0 Newton force is applied, which is the thickness of the layer of aggregates at the indicated compression force, and is the Dry Aggregate Strength. The measurement is again performed on the aggregate composition in the same manner except that immediately prior to taring the normal force, sufficient water is added to the edge of the gap to wick into the gap being careful not to allow aggregates to be pushed out from under the gap. After a 60 second pause, the measurement continues as before, and the result obtained with a 5.0 Newton force is the Wet Aggregate Strength. The Aggregate Wet Strength Ratio is expressed as the Wet Aggregate Strength divided by the Dry Aggregate Strength, and multiplied by 100%.

### IV. Examples

### Example 1

Zinc pyrithione is obtained from Arch Chemicals as a 49% active suspension of FPS zinc pyrithione, such as Zinc OMADINE® Zinc OMADINE® can include trace components that can provide benefits to FPS zinc pyrithione, such as stability. For example, FPS zinc pyrithione includes about 0.25% of carboxymethyl cellulose, by weight of the FPS zinc pyrithione. And further, FPS zinc pyrithione can include from about 0.1% to about 2.5% sodium polynaphthalenesulfonate, by weight of the FPS zinc pyrithione. Additionally, the FPS zinc pyrithione particles can have a mean diameter of about 0.75 micron as determined by light scattering. Table 1 provides information relating to Comparative Example 1, illustrating traditional FPS zinc pyrithione.

TABLE 1

| | Added | | ZPT:Added | |
|---|---|---|---|---|
| | Polymer | Type | Polymer Ratio | Drying Method |
| Comparative | None | None | None | None |
| Example 1 | | | | |

### Examples 2-6

To prepare a tray-dried zinc pyrithione-polymer aggregate, first, zinc pyrithione is obtained from Arch Chemicals as a 49% active suspension of FPS Zinc OMADINE®. Comparative Examples 2A-2C are prepared by adding the zinc pyrithione to an aluminum foil boat and drying in an oven, at a temperature of from about 60° C. to about 70° C., until dry. For Inventive Examples 3A-3C, 4A-4C, 5A-5C, and 6A-6C, binders, such as polymers, are obtained directly from their respective suppliers. Aqueous solutions of polymers are prepared and subsequently mixed with a zinc pyrithione aqueous suspension in relative proportions to deliver desired dry-weight based ratios of zinc pyrithione to polymer. The mixtures are then transferred to trays and dried in an oven, at a temperature of about 45° C., until solid. Once thoroughly dry, aggregate material, for Comparative Examples 2A-2C and Inventive Examples 3A-3C, 4A-4C, 5A-5C, and 6A-6C, is then removed from the tray and mechanically broken into small particles. Resulting fractions are then sieved using U.S. Standard Sieves to yield particle size fractions. Table 2 provides information relating to Comparative Examples 2A-2C and Inventive Examples 3A-3C, 4A-4C, 5A-5C, and 6A-6C.

TABLE 2

| | Added | | ZPT:Added Polymer | | |
|---|---|---|---|---|---|
| | Polymer | Type | Ratio | Aggregate or Particle Size | |
| Comparative Example 2A | None | None | None | <600 | μm |
| Comparative Example 2B | None | None | None | 600-850 | μm |
| Comparative Example 2C | None | None | None | 850 μm to about 1,500 μm | |
| Inventive Example 3A | AM:Triquat | Cationic | 99:1 | <600 | μm |
| Inventive Example 3B | AM:Triquat | Cationic | 99:1 | 600-850 | μm |
| Inventive Example 3C | AM:Triquat | Cationic | 99:1 | 850 μm to about 1,500 μm | |
| Inventive Example 4A | AM:Triquat | Cationic | 90:10 | <600 | μm |
| Inventive Example 4B | AM:Triquat | Cationic | 90:10 | 600-850 | μm |
| Inventive Example 4C | AM:Triquat | Cationic | 90:10 | 850 μm to about 1,500 μm | |
| Inventive Example 5A | AM:Triquat | Cationic | 80:20 | <600 | μm |
| Inventive Example 5B | AM:Triquat | Cationic | 80:20 | 600-850 | μm |
| Inventive Example 5C | AM:Triquat | Cationic | 80:20 | 850 μm to about 1,500 μm | |
| Inventive Example 6A | HPMC | Non-ionic | 99:1 | <600 | μm |
| Inventive Example 6B | HPMC | Non-ionic | 99:1 | 600-850 | μm |

| | | | | |
|---|---|---|---|---|
| Inventive Example 6C | HPMC | Non-ionic | 99:1 | 850 μm to about 1,500 μm |

*Zinc pyrithione used in the above examples was FPS zinc pyrithione obtained from Arch Chemicals.

### Examples 7-10

To prepare a spray-dried zinc pyrithione-polymer aggregate, first, zinc pyrithione is obtained from Arch Chemicals as a 48% active suspension of FPS Zinc OMADINE®. Binders, such as polymers, are obtained directly from their respective suppliers. Aqueous solutions of polymers are prepared and subsequently mixed with a zinc pyrithione aqueous suspension in relative proportions to deliver desired dry-weight based ratios of zinc pyrithione to polymer. The zinc pyrithione-polymer mixture is then spray dried using a Büchi Mini Spray Dryer B290 with an inlet temperature of 200° C. and an outlet temperature of 100° C. The slurry flow rate is controlled by adjusting the peristaltic pump control to 35% of maximum pump speed. The compressed air flow rate for the feed dispersion is set to approximately 600 L/hr. The spray-dried zinc pyrithione-polymer aggregates are observed to have particle sizes of about 10 microns to about 100 microns by light microscopy with an average diameter of about 60 microns, while being comprised of distinct primary particle subunits which are the original FPS particles. The aggregates are spherical. Table 3 provides information relating to Comparative Example 7 and Inventive Examples 8-10A. Inventive Example 9 cites Guar hydroxypropyltrimonium chloride (Guar HPTC). Inventive Example 10B, listed in Table 7, is prepared with hydroxypropyl cellulose.

TABLE 3

| | Added | | ZPT:Added Polymer | |
|---|---|---|---|---|
| | Polymer | Type | Ratio | Drying Method |
| Comparative Example 7 | None | None | None | Spray Drying |
| Inventive Example 8 | AM:Triquat | Cationic | 99:1 | Spray Drying |
| Inventive Example 9 | Guar HPTC | Cationic | 99:1 | Spray Drying |
| Inventive Example 10A | HPMC | Non-ionic | 99:1 | Spray Drying |

*Zinc pyrithione used in the above examples was FPS zinc pyrithione obtained from Arch Chemicals.

### Examples 11-15

For Comparative Examples 11 and 12, a bar soap is prepared comprising spray-dried zinc pyrithione, and for Inventive Examples 13-15, a bar soap is prepared comprising a spray-dried zinc pyrithione-polymer aggregate. Soap noodles, made via a conventional process involving a crutching step and vacuum drying step, are blended with one of the spray-dried zinc pyrithione or the spray-dried zinc pyrithione-polymer aggregate in an amalgamator, depending on the formulation. A soap blend is then processed through conventional milling, plodding, and stamping steps to yield finished bar compositions. Example compositional information with respect to some comparative (Examples 11-12) and inventive (Examples 13-15) formulations can be found in Table 4.

TABLE 4

| | | | Inventive Ex. 13: | Inventive Ex. 14: | Inventive Ex. 15: |
|---|---|---|---|---|---|
| | | | 0.5% ZPT Bar with | 0.5% ZPT Bar with | 0.5% ZPT Bar with |
| | | Comparative Ex. 12: | Non-ionic Polymer | Non-ionic Polymer | Cationic Polymer |
| | Comparative Ex. 11: | 0.5% ZPT Bar | (Tray-dried ZPT- | (Spray-dried ZPT- | (Spray-dried ZPT- |
| | 0.5% ZPT Bar (FPS | (Spray-dried ZPT), | Polymer Aggregate), | Polymer Aggregate), | Polymer Aggregate), |
| Ingredient | ZPT), wt. % | wt. % | wt. % | wt. % | wt. % |
| Sodium tallowate | 67.64 | 67.64 | 67.64 | 67.64 | 67.64 |
| Sodium palm | 17.26 | 17.26 | 17.26 | 17.26 | 17.26 |
| kernelate | | | | | |
| Water | QS | QS | QS | QS | QS |
| Sodium chloride | 0.62 | 0.62 | 0.62 | 0.62 | 0.62 |
| Comparative Ex. 1 | 0.5 | — | — | — | — |
| Inventive Ex. 6A | — | — | 0.5 | — | — |
| Inventive Ex. 10A | — | — | — | 0.5 | — |
| Comparative Ex. 7 | — | 0.5 | — | — | — |
| Inventive Ex. 8 | — | — | — | — | 0.5 |
| Glycerin | 0.3 | 0.3 | 0.3 | 0.3 | 0.3 |
| Palm kernel acid | 0.21 | 0.21 | 0.21 | 0.21 | 0.21 |
| Tallow acid | 0.14 | 0.14 | 0.14 | 0.14 | 0.14 |
| EDTA | 0.05 | 0.05 | 0.05 | 0.05 | 0.05 |
| Minors-By products | 0.28 | 0.28 | 0.28 | 0.28 | 0.28 |

*Zinc pyrithione used in the above examples was FPS zinc pyrithione obtained from Arch Chemicals.

### Example 16

A micellar body wash is prepared comprising a dried zinc pyrithione-polymer aggregate. Surfactants are added with excess water and stirred until homogenous. A polymer is added from a 20% solution, and then all other ingredients are added, except for salt and Ethylene glycol distearate (EGDS), which are added and stirred until homogeneous. EGDS is separately prepared by precipitating from a hot solution as a concentrate with SLS and added as a concentrated premix. The pH is adjusted to 6.0. Sodium chloride is added last to obtain a Brookfield viscosity of 9,000 cP at 2.0 l/seconds shear rate. Table 5 shows an example formulation of a micellar body wash.

TABLE 5

| Ingredient | Amount, wt. % |
|---|---|
| Sodium laureth-1 sulfate | 9.50 |
| Cocamidopropyl betaine | 1.50 |
| Citric acid | 0.34 |
| Polyquaternium 76 | 0.30 |
| EGDS | 3.50 |
| Inventive Example 8 | 1.00 |
| Zinc carbonate | 1.50 |
| Sodium Chloride | 1.25 |
| Fragrance | 1.00 |
| Preservatives | 0.41 |
| Water | QS |

### Example 17

The cleansing phase is prepared by conventional formulation and mixing techniques. The cleansing phase is prepared by first adding water, skin benefit components, and thickeners into a mixing vessel and agitating until a homogeneous dispersion is formed. Then the following ingredients are added in the following sequence: surfactants, disodium EDTA, preservatives, half of a total amount of sodium chloride and all other preservatives and minors, except a fragrance and a withheld half of the sodium chloride. The ingredients are maintained at ambient temperature while agitating the mixing vessel. In a separate vessel, structuring polymers are pre-wet with the fragrance and added to the mixing vessel at the same time as the remaining sodium chloride while agitating. A dried zinc pyrithione-polymer aggregate and soybean oil are then added. The mixture is agitated until homogeneous and then pumped through a static mixing element to disperse any polymer lumps to complete the batch. Table 6 shows an example formulation of a structured surfactant body wash.

TABLE 6

| Ingredients | Amount, wt. % |
|---|---|
| Water, distilled | QS |
| Glycerin | 0.80 |
| Guar hydroxypropropyl-trimonium chloride (N-Hance | 0.70 |
| 3196, Aqualon) | |
| PEG 90M (Polyox WSR 301, Amerchol Corp) | 0.20 |
| Citric acid | 0.40 |
| Sodium Trideceth Sulfate, Sodium Laurampho-acetate, | 23.70 |
| Cocamide MEA (Miracare SLB-365, Rhodia, Inc.) | |
| Fragrance | 1.40 |
| Soybean oil | 5.00 |
| Sodium chloride | 3.50 |
| Preservatives | 0.45 |
| Inventive Example 8 | 0.50 |
| Final pH (adjust using NaOH or citric acid) | 6.2 |
| Zero shear viscosity | 6530 Pascal- |
| | seconds |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm” Also, percentages provided herein are expressed on a water-free basis, except where indicated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of enhancing the deposition of zinc pyrithione, comprising combining zinc pyrithione with a polymer and drying the zinc pyrithione polymer combination to form an aggregate.

2. The method of claim 1, wherein the zinc pyrithione is in the form of a suspension prior to combination with the polymer.

3. The method of claim 1, wherein the polymer is in the form of a solution prior to combination with the zinc pyrithione.

4. The method of claim 1, wherein the zinc pyrithione-polymer aggregate has a moisture content of about 25% or less, by weight of the aggregate.

5. The method of claim 1, wherein the zinc pyrithione-polymer aggregate comprises from about 0.1% to about 20% of polymer, by weight of the aggregate, of the polymer, and from about 80% to about 99.9%, by weight of the aggregate, of zinc pyrithione.

6. The method of claim 1, wherein the zinc pyrithione polymer combination is spray dried to form the aggregate.

7. The method of claim 1, wherein the polymer comprises cellulose, xanthan gum, polyvinyl alcohol, ethylenevinyl acetate, silicone, acrylamide, guar, polyisobutylene, a material selected from the group consisting of poly(isobutylene), a copolymer comprising poly(isobutylene), poly(ethylene), a copolymer comprising poly(ethylene), poly(propylene), a copolymer comprising poly(propylene), poly(butadiene), a copolymer comprising poly(butadiene), poly(isoprene), a copolymer comprising poly(isoprene), hydrogenated poly(butadiene), a copolymer comprising hydrogenated poly(butadiene), hydrogenated poly(isoprene), a copolymer comprising hydrogenated poly(isoprene), poly(alpha-olefin), a copolymer comprising poly(alpha-olefin), petrolatum, microcrystalline wax, or combinations thereof.

8. The method of claim 1, wherein the dried zinc pyrithione-polymer aggregate comprises a hydrophobic polymer.

9. The method of claim 1, wherein the dried zinc pyrithione-polymer aggregate comprises an acrylamide-based polymer.

10. The method of claim 1, wherein the dried zinc pyrithione-polymer aggregate comprises a hydrophobic polymer.

11. The method of claim 1, wherein the dried zinc pyrithione-polymer aggregate comprises:
a) from about 1% to about 10%, by weight of the dried zinc pyrithione-polymer aggregate, of a polymer; and
b) from about 90% to about 99%, by weight of the dried zinc pyrithione-polymer aggregate, of zinc pyrithione.

12. The method of claim 1, wherein the dried zinc pyrithione exhibits a compressive strength thickness of from about 100 microns to about 300 microns.

13. The method of claim 1, wherein the polymer comprises an acrylamide triquat copolymer.

14. The method of claim 1, wherein the polymer comprises a cationic guar.

15. The method of claim 1, wherein the zinc pyrithione-polymer aggregate has a moisture content of about 20% or less, by weight of the aggregate.

16. The method 1, wherein the zinc pyrithione-polymer aggregate has a moisture content of about 15% or less, by weight of the aggregate.
